(19) United States
(12) Patent Application Publication
(10) Pub. No.: US 2003/0175237 A1
(43) Pub. Date: Sep. 18, 2003

(54) Method for the reduction of fasting plasma glucose and hemoglobin AIC levels

(76) Inventors: William L. Bailey, Mendham, NJ (US); Mark A. Wingertzahn, Stroudsburg, PA (US); Jeffrey T. Jewell, Morrisville, NC (US)

Correspondence Address: BRUCE D. RADIN, Budd Larner Rosenbaum,, Greenberg & Sade, P.C., 150 John F. Kennedy Parkway, Short Hills, NJ, 07078, US

(21) Appl. No.: 10/375,202

(22) Filed: Feb. 26, 2003

Related U.S. Application Data
(60) Provisional application No. 60/359,890, filed on Feb. 26, 2002.

(51) Int. Cl. A61K 31/785; A61K 31/225; A61K 31/401; A61K 31/366

(52) U.S. Cl. 424/78.36; 514/423; 514/460; 514/548

(57) ABSTRACT

A method of reducing levels of fasting plasma glucose and/or hemoglobin A_{1C} in patients by the administration of an aliphatic amine polymer, such as colesevelam HC1, either alone or in combination with a statin. In particular, the invention reduces LDL-cholesterol, FPG and HbA_{1c} in patients with or without glucose intolerance. Also, the invention provides a method of treating hyperglycemia and prevent or delay associated microvasular complications in patients with impaired fasting glucose, T2DM and insulin resistance.

### STATEMENT OF RELATED APPLICATION

This application claims the benefit of priority of U.S. Provisional Application No. 60/359,890, filed on Feb. 26, 2002.

The entire teaching of the above-referenced application is incorporated herein by reference.

### FIELD OF THE INVENTION

The present invention relates to a method of reducing levels of fasting plasma glucose and/or hemoglobin A_{1c} by the administration of an aliphatic amine polymer, such as colesevelam HC1, either alone or in combination with a statin.

### BACKGROUND OF THE INVENTION

Several polymeric materials have been described for lowering LDL-cholesterol levels. Some such materials are nonsystemic, which means that the body does not absorb them and they are eliminated without traveling to the liver, kidneys or other organs. These materials work in the intestine, where they bind to bile acids and are removed through the normal digestive process. This causes a chain of events leading to LDL-cholesterol lowering.

Examples of these materials include colestipol HC1 and cholestyramine. Cholestyramine, a polystrene/divinylbenzene ammonium ion exchange resin, is unpalatable, gritty and highly constipating. Newer non-absorbed polymeric, lipid lowering agents include aliphatic amine polymer resins and aliphatic amine polymer resins that are alkylated, such as, for example, colesevelam HC1.

Colesevelam HC1 also offers several benefits over traditional bile-acid binding agents such as cholestyramine and colestipol HC1, specifically related to dosage forms and amount, as well as improved drug:drug interactions and side effect profiles, i.e., less constipating. Currently, dosage forms of these traditional bile-acid binding agents include unpalatable powder and large 1 gram tablets requiring up to 16 grams/day for a certain level of reduction in serum cholesterol. Colesevelam HC1, by contrast, is marketed as a 625 mg tablet (which is approximately ⅓ the size of a colestid tablet) and a typical dose is 3.8 g (6 tablets) per day to achieve similar reductions in serum cholesterol seen with either cholestyramine or colestipol HC1. See e.g., JAMA 1984:251 pp 365-374. The smaller tablet size and number, as well as the absence of a gritty consistency, make colesevelam HC1 a more convenient and palatable formulation. Additionally, colesevelam HC1 does not show significant drug: drug interactions with agents that have been previously shown to interact with conventional bile acid binding agents (e.g. digoxin, warfarin, valproic acid, lovastatin, metoprolol and quinidine). Colesevelam HC1, in clinical trials, has also demonstrated a low incidence of gastrointestinal disturbance, a side effect that is common to traditional bile acid binding agents.

Previous work on traditional bile-acid binding agents such as cholestyramine showed that the administration of cholestyramine in diabetics decreased LDL-cholesterol levels as well as fasting plasma glucose levels as compared with a placebo, although changes in glycosylated hemoglobin (HbA_{1c}) levels were not statistically significant. See Garg & Grundy, Ann Intern. Med. 1994, 121(6):416-422. Unexpectedly, it has been found that aliphatic amine polymer resins, such as colesevelam HC1, either alone or in combination with a statin, reduces fasting plasma glucose (FPG) and/or hemoglobin A_{1c} (HbA_{1c}).

### SUMMARY OF THE INVENTION

The present invention relates to the discovery that colesevelam HC1, either alone or in combination with a statin, in addition to reducing LDL-cholesterol levels, also reduces fasting plasma glucose levels and/or hemoglobin A_{1c} levels. It is accordingly an object of the present invention to provide a method for lowering fasting plasma glucose levels and/or hemoglobin A_{1c} levels in patients, such as diabetics, who may also require reduction in LDL-cholesterol levels.

Patients with abnormal glucose tolerance, manifesting as either impaired fasting glucose (IFG) or overt type 2 diabetes mellitus (T2DM), often times also have dyslipidemia requiring multiple drugs to treat these seemingly distinct biochemical abnormalities. It is thus one object of the present invention to reduce LDL-cholesterol, FPG and HbA_{1c} in patients with or without glucose intolerance. It is a further object of the present invention to provide a method of treating hyperglycemia and prevent or delay associated microvasular complications in patients with impaired fasting glucose, T2DM and insulin resistance.

Although some hypoglycemic agents can also improve lipid abnormalities associated with IFG and T2DM (e.g. thiazolidinediones, metformin, etc), there is a need for agents that can accomplish this without being systemically absorbed or metabolized. This could become of particular importance in patients on multiple drugs that are metabolized by similar pathways, leading to potential deleterious drug toxicities and/or side effects, particularly in individuals with compromised organ function. It is thus another object of the present invention to provide for a method that affects both lipid and glucose metabolism in order to offer several benefits over currently available therapies including: reduction in number of systemic drug therapies utilized to treat these biochemical abnormalities, reduction in incidence of drug toxicities, increased compliance and reduced drug costs.

Other features and advantages will be apparent from the following description of the preferred embodiments thereof and from the claims.

### DETAILED DESCRIPTION OF THE INVENTION

The aliphatic amine polymer resin of the present invention can be any of the aliphatic amine resins described in U.S. Pat. Nos. 5,496,545; 5,667,775; 5,624,963; 5,703,188; 5,679,717; 5,693,675; 5,607,669; 5,618,530; 5,487,888; 5,917,007; 5,919,832; 5,981,693; and 5,702,696, each; of which is hereby incorporated herein by reference in its entirety. Other suitable aliphatic amine polymers are disclosed in U.S. Pat. Nos. 6,034,129 and 5,840,766 each of which is hereby incorporated by reference in its entirety. The alkylated aliphatic amine polymer can be any of these as described in U.S. Pat. Nos. 5,624,963; 5,679,717 and 5,607,669, each of which is hereby incorporated by reference in its entirety. In a particularly preferred embodiment, the aliphatic amine polymer is polyallylamine, alkylated polyallylamine, polyvinylamine, poly (diallylamine) or poly (ethyleneimine) or a salt thereof with a pharmaceutically acceptable acid. The aliphatic amine polymer is optionally substituted at one or more nitrogen atoms with an alkyl group or a substituted alkyl group such as a trialkylammonioalkyl group. The aliphatic amine polymer can optionally be cross-linked, for example via a multifunctional monomer or a bridging group which connects two amino nitrogen atoms from two different polymer strands. In a preferred embodiment, the aliphatic amine polymer resin is hydrated. In the most preferred embodiment, the aliphatic amine polymer is a poly (allylamine hydrochloride) crosslinked with epichorohydrin and alkylated with 1-bromodecane and (6-bromohexyl)-trimethylammonium bromide also referred to as colesevelam or colesevelam HC1 (trademark WelChol®, Sankyo Co. Ltd. Corp.). Colesevelam HC1, described in, e.g., U.S. Pat. Nos. 5,607,669, 5,679,717, 6,066,678 and 6,225,355 each of which is incorporated herein by reference in its entirety, is among a new class of ion exchange resins, specifically engineered to have specificity, affinity and high capacity to bind bile acids, that has improved bile acid sequestration properties and little to no grittiness, thereby improving the palatability of the composition. Colesevelam HC1, either alone or in combination with a HMG-CoA reductive inhibitor (statin) has been shown to reduce LDL-cholesterol levels, decrease total cholesterol, increase HDL cholesterol and increase APOA.

In studies involving colesevelam HC1, plasma glucose decreased in the two high dose colesevelam HC1 groups. In an analysis of patients with diabetes (n=13), serum glucose fell from 140 mg/dL on diet alone to 122 mg/dL when patients were given colesevelam HC1 3.8 or 4.5 g/d (p<0.01). Similar effects were seen in an analysis of the integrated safety data.

Specifically, glucose was measured as a safety laboratory at weeks −4, 0, 12 and 24. There was a significant group effect for serum glucose (p=0.03) for change from day 0 to day 168 for all patients. Decreases from day 0 to day 168 of 2.8 and 2.2 mg/dL in the colesevelam 3.8 and 4.5 g/d groups, respectively, were statistically significant (p<0.01).

The effect of colesevelam HC1 was reviewed on serum glucose in patients with diagnosed diabetes and also included patients with undiagnosed diabetes (meeting ADA criteria for fasting glucose). HgbA_{1c} was not measured as a safety lab value. Because of the small numbers, the two lower doses of colesevelam HC1 (2.3 and 3.0 g/d) and the two higher doses of colesevelam HC1 (3.8 and 4.5 g/d) were pooled. Baseline was taken as the average of values at weeks −4 and 0, and endpoint was taken as the average of values at 12 and 24 weeks. While there were small changes in serum glucose for the placebo and low dose groups, serum glucose in the high dose group fell an average 12% from 140 to 122 mg/dL (p=0.005). When analysis was confined to diagnosed diabetics (n=8), the decrease in serum glucose in the high dose group was 13%. The majority of diabetics showed decreases in serum glucose. Analysis of the patients taking colesevelam HC1 suggests that diabetics had improvements in glycemic control similar in magnitude to those shown for cholestyramine, but at much lower doses (@4.5 g vs. 16 g/d). The fact that fasting plasma glucose levels are lowered over a period of time is also indicative that Hemoglobin A_{1c} levels will also be lowered.

Therapeutically effective monotherapy dosages of colesevelam HC1 can be on the order of 3 to 6 grams per day and are typically on the order of 3.8 to 4.5 grams per day. Therapeutically effective combination dosages of colesevelam HC1 with a statin are typically on the order of 2.3 to 3.8 grams per day. The method of the present invention provides for the administering of a therapeutically effective amount of colsevalem HC1 or a pharmaceutically acceptable salt thereof to a patient to reduce plasma glucose levels and/or hemoglobin A_{1c}, either alone or in combination with other lipid lowering medications, such as statins. Such statins can include, for example, atorvastatin, simvastatin, and lovastatin. In one example, a combination of 3.8 g/day of colesevelam HC1 and 10 mg of atorvastatin can be used as a combination therapy. In another example, 3.8 g/day of colesevelam HC1 and 10 mg/day of simvastatin can be used as a combination therapy. In a further example, 2.3 g/day of colesevelam HC1 and 10 mg/day of lovastatin can be used as a combination therapy.

It will be apparent to those skilled in the art that various modifications and variations can be made to the present invention without departing from that spirit or scope thereof and that such modifications and variations can be performed using routine experimentation. Thus, it is intended that the present method covers the modifications and variations thereof provided they were within the scope of the appended claims and their equivalents.

What is claimed is:

1. A method of lowering fasting plasma glucose and hemoglobin A_{1c} in a patient in need thereof, the method comprising administering to the patient a therapeutically effective amount of an aliphatic amine polymer or a pharmaceutically acceptable salt thereof.

2. A method of claim 1 wherein the aliphatic amine polymer or pharmaceutically acceptable salt thereof is co-administered with a statin.

3. A method of claim 1 wherein the aliphatic amine polymer is colesevelam HC1.

4. A method of claim 2 wherein the aliphatic amine polymer is colesevelam HC1.

5. A method of claim 3 wherein the amount of colesevelam HC1 is from about 2.3 grams to about 4.5 grams per day.

6. A method of claim 4 wherein the amount of colesevelam HC1 is from about 2.3 grams to about 4.5 grams per day;

7. A method of claim 3 wherein the statin is atorvastatin, simvastatin or lovastatin.

8. A method of claim 4 wherein the statin is atorvastatin, simvastatin or lovastatin.

9. A method of claim 5 wherein the statin is atorvastatin, simvastatin or lovastatin.

10. A method of claim 6 wherein the statin is atorvastatin, simvastatin or lovastatin.

11. A method of lowering fasting plasma glucose in a patient in need thereof, the method comprising administering to the patient a therapeutically effective amount of an aliphatic amine polymer or a pharmaceutically acceptable salt thereof.

12. A method of claim 11 wherein the aliphatic amine polymer or pharmaceutically acceptable salt thereof is co-administered with a statin.

13. A method of claim 11 wherein the aliphatic amine polymer is colesevelam HC1.

14. A method of claim 12 wherein the aliphatic amine polymer is colesevelam HC1.

15. A method of claim 13 wherein the amount of colesevelam HC1 is from about 2.3 grams to about 4.5 grams per day;

16. A method of claim 14 wherein the amount of colesevelam HC1 is from about 2.3 grams to about 4.5 grams per day;

17. A method of claim 13 wherein the statin is atorvastatin, simvastatin or lovastatin.

18. A method of claim 14 wherein the statin is atorvastatin, simvastatin or lovastatin.

19. A method of claim 15 wherein the statin is atorvastatin, simvastatin or lovastatin.

20. A method of claim 16 wherein the statin is atorvastatin, simvastatin or lovastatin.

21. A method of lowering hemoglobin A_{1c} in a patient in need thereof, said method comprising administering to the patient a therapeutically effective amount of an aliphatic amine polymer or a pharmaceutically acceptable salt thereof.

22. A method of claim 21 wherein the aliphatic amine polymer or pharmaceutically acceptable salt thereof is co-administered with a statin.

23. A method of claim 21 wherein the aliphatic amine polymer is colesevelam HC1.

24. A method of claim 22 wherein the aliphatic amine polymer is colesevelam HC1.

25. A method of claim 23 wherein the amount of colesevelam HC1 is from about 2.3 grams to about 4.5 grams per day.

26. A method of claim 24 wherein the amount of colesevelam HC1 is from about 2.3 grams to about 4.5 grams per day.

27. A method of claim 23 wherein the statin is atorvastatin, simvastatin or lovastatin.

28. A method of claim 24 wherein the statin is atorvastatin, simvastatin or lovastatin.

29. A method of claim 25 wherein the statin is atorvastatin, simvastatin or lovastatin.

30. A method of claim 26 wherein the statin is atorvastatin, simvastatin or lovastatin.
